(19) United States
(12) Patent Application Publication
(10) Pub. No.: US 2008/0019917 A1
(43) Pub. Date: Jan. 24, 2008

(54) Compositions Comprising Cells and Magnetic Materials for Targeted Delivery

(75) Inventor: Andrew Pacey, Herts (GB)

Correspondence Address: NEEDLE & ROSENBERG, P.C., SUITE 1000, 999 PEACHTREE STREET, ATLANTA, GA, 30309-3915, US

(73) Assignee: MAGNET ATTRACTION LIMITED

(21) Appl. No.: 10/592,288

(22) PCT Filed: Mar. 14, 2005
(86) PCT No.: PCT/GB05/01028

§ 371 Date: Jun. 18, 2007

(30) Foreign Application Priority Data
Mar. 12, 2004 (GB) 0405552.1

(51) Int. Cl. A61B 5/05; A61F 2/00; A61P 17/02

(52) U.S. Cl. 424/9.3; 623/1.15

(57) ABSTRACT

The present invention provides a method in which a formulation comprising cells and a magnetic material is administered to a subject and part of the body of said subject is subjected to a magnetic field, as well as a kit for use in this method. A medical device for use in this method is also provided.

The present invention relates to therapeutic or cosmetic formulations which may be targeted, in vivo, to regions of the animal body, in particular to formulations comprising nucleic acid such as cells.

The point at which a formulation is administered, e.g. orally or systemically by injection into a peripheral blood vessel may be remote from the region of the body on which the formulation is desired to act. In such cases it may be desirable to target the formulation to the region of the body at which it is required, i.e. the target area.

In other instances, administration may occur at or around the target area, e.g. by subcutaneous injection or via a catheter. In such cases it may be desirable to ensure that the formulation remains substantially at or in the close vicinity of the site of administration, i.e. to inhibit dispersal of the formulation away from the target area.

Clearly there are advantages if the formulation can be targeted in some way in terms of a potential reduction in undesirable side-effects, toxic effects and the administration of a lower dose overall. Targeting may be achieved passively, for example with liposomes having incorporated therein, proteinaceous affinity ligands for a binding partner (receptor) found on target cells. ‘Targeting’ as used herein refers both to the case where the formulation is administered at a remote position from the target area and travels there through the body and where movement of the formulation away from the point of administration is inhibited.

The desire for specific and controlled medical treatments or cosmetic applications means there is a continuing need for alternative or improved methods of drug delivery and more generally for the delivery of formulations, particularly formulations comprising cells, for therapeutic, cosmetic or other purposes. These objectives are addressed by the present invention which provides, in one aspect, a method in which a therapeutic or cosmetic formulation comprising nucleic acid and a magnetic material is administered to a subject, part of the body of said subject (which it is desired to treat with the formulation) being subjected to a magnetic field.

In a preferred embodiment the present invention provides a method in which a formulation comprising cells and a magnetic material is administered to a subject and part of the body of said subject is subjected to a magnetic field.

The magnetic field will typically be in close proximity to the target area e.g. no more than 10 cm, preferably no more than 5 cm, more preferably no more than 2 cm from it, but preferably it will overlap at least partially with it. Most preferably, all or at least a large proportion of the magnetic field will overlap with the target area.

By “target area” is meant the part of the subject's body at which the presence of the formulation of the present invention is required. In the case of medical treatment, the target area may be all or part of the area which is affected by a medical condition. In most cases it will be sufficient to target the formulations of the present invention to just one part of the affected area. For example, in the case of a tumour the target area may be only a small part of the tumour. Without wishing to be bound by theory, it is thought that by targeting e.g. T lymphocytes to part of a tumour, an immune response is elicited in the subject's body which may eliminate the entire tumour.

A magnet will be used to generate the magnetic field. Any kind of magnet may be used. Electromagnets are preferred, providing a greater ease of utility in switching on and off and controlling the magnetic field.

The magnet will preferably be substantially in direct or indirect physical contact with the subject during application of the magnetic field. The magnet may be coated with a protective layer and/or covered with e.g. a sleeve or mould and in such instances it will be this coat or cover which will preferably be in direct or indirect physical contact with the subject. Thus, the magnet will not be further than 50 cm from the subject, preferably no more than 20, 10 or 5 cm from the subject, more preferably no more than 2 cm and even more preferably no more than 1 cm. Most preferably, the distance between the magnet and the subject is no more than a few millimeters. Methods in which the magnet has no physical contact with the subject and is at a distance of more than 50 cm from the subject do not form part of the present invention.

The magnet may be internal or external to the subject. External magnets may be placed on the skin, directly or indirectly e.g. via a pad or dressing. The presence of e.g. a layer of clothing between the magnet and the subject will typically be acceptable. External magnets will be preferred when the target area is at or close to the subject's body surface and/or when prolonged application of a magnetic field is desired.

Internal magnets may be introduced into the subject e.g. as part of a catheter, stent, needle or the like. All internally introduced magnets can be considered to be in direct contact with the subject even if they are coated or covered in some manner. Internal magnets are preferred when the target area is not at the surface of the body of the subject, e.g. when the target is an internal organ.

The magnetic field may start to be applied before, during or after administration of the formulation to the subject. Thus the methods of the invention comprise two steps:

(a) administration to a subject of a formulation comprising cells and a magnetic material; and

(b) subjecting part of the body of said subject to a magnetic field; these two steps being performed simultaneously or sequentially. Typically if administration of the magnetic field commences prior to administration it will continue (or recommence) during or after administration. Thus if there is no period during which the subject is being subjected to a magnetic field and being administered with the formulation, i.e. the two steps are performed entirely sequentially, then the magnetic field is applied as the second step. Preferably, the field is applied no longer than 1 hour after administration of the formulation, more preferably no longer than 20 minutes and even more preferably no longer than 10 minutes after administration. Most preferably, it is applied substantially immediately after administration.

Depending on the mode of administration and the location of the magnet, it may be necessary to perform the administration step before the magnetic field is applied. For example, if the magnet is located in or on the administration device such as a catheter or stent, application of the magnetic field during administration could interfere with the administration, e.g. leading to clumping of the formulation or retention of the formulation inside or on the device. The skilled man will be able to use common sense to determine whether the two steps should be carried out simultaneously or sequentially.

Application of the magnetic field should be continued for a suitable period of time after administration of the formulation which will vary depending on the therapeutic purpose and the cell type. Without wishing to be bound by theory, we postulate that cell-to-cell interactions contribute to keeping a cell in its location. Thus, if a formulation containing a cell is targeted to a target area and retained at that area through a magnetic field, after a suitable period of time sufficient cell-to-cell interactions will have formed to ensure that the introduced cell remains at the target site even when the magnetic field ceases. Suitable periods of time may vary but will typically be at least 20 minutes, preferably at least 30 minutes, e.g. 30-45 minutes.

The magnetic field is preferably applied for a minimum of 20 minutes, preferably at least 30 minutes, more preferably at least 1 hour. It may be applied for longer, e.g. for more than 2, 3, 4 or 5 hours, e.g. up to 24 hours or for several days, especially when a convenient magnet that does not cause patient discomfort is used.

In instances where the site of administration is remote from the target area the cells need to be attracted to the target area and then retained at the target area. The skilled person will be able to determine, if required, when the cells have reached the target area, e.g. through nuclear magnetic resonance (NMR) scanning. Application of the magnetic field to target the cells should not produce any deleterious effects and increasing the length of the application of the magnetic field should therefore be unproblematic.

The strength of the magnetic field required will inter alia be dependent on the size and magnetic strength of the magnetic material used in the formulation. Thus, as a general rule, smaller magnetic particles require a stronger magnetic field than larger particles. The skilled man will be aware of the safety margin within which he can operate, i.e. he will know not to use dangerously high magnetic fields. He will easily be able to determine what magnetic strength to use in the methods of the present invention.

Suitable magnetic fields produced by external permanent magnets are known in the art. For Example, there are many commercially available magnets for use in “magnetic therapy” (which uses magnets to treat a variety of complaints, but does not use formulations comprising magnetic materials). Examples of suitable permanent magnets include those with a flux range from N25-N48. For electromagnets, currents in the range of about 1 mA to 13 A may be suitable, preferably between 10 mA and 13 A.

Suitable magnetic field may also be determined experimentally. For example, cells containing magnetic material are injected into an experimental animal or a tissue sample thereof, a magnetic field is applied to a target site and after a suitable period of time (e.g. 30 min) the area surrounding the target site is sectioned and examined under the microscope to determine whether and to what extent the cells have migrated towards, or been retained at (as the case may be) the target area. The injected cells will be easily recognised because they contain the magnetic particles. The cells may be injected at different distances from the magnet to determine over what distance the magnet is effective. An electromagnet may be used to create different magnetic field strengths and this will allow the skilled person to test which strength is appropriate.

The strength of a magnet may also be tested by placing a magnet onto a gelatin mixture loaded with ferrite fillings which has been allowed to set.

Formulations suitable for in vivo administration which contain nucleic acid (e.g. in a vector or cell) are well known in the art. They will typically be liquid formulations comprising the cells, vector or nucleic acid and a physiologically acceptable liquid carrier.

The therapeutic formulation will typically comprise cells, those cells containing the aforementioned nucleic acid and also containing or being conjugated with magnetic material. Preferably the cells contain the magnetic material. Any cell which can be of therapeutic benefit to the subject is contemplated. The benefit may arise from the normal properties and functions of the cell, e.g. wherein the cell is a lymphocyte, e.g. a T lymphocyte. By way of example, the cells may be dendritic cells, T-lymphocytes including cytoxtic T-lymphocytes or other immunologically active cells. Stem cells are particularly preferred.

The cell may be acting as an in situ protein producer, wherein the protein is of therapeutic benefit. Any expressed protein may be the product of the cell's native nucleic acid or the product of heterologous nucleic acid which the cell has been modified to contain. Cells which have not been genetically manipulated to contain a heterologous gene are particularly preferred in the context of the present invention. Thus the cell may act as a convenient carrier of a therapeutic agent, in particular of a nucleic acid which encodes a therapeutic agent.

The formulations may be directed to any area that may benefit from cell therapy. Examples include any area in need of regeneration such as a damaged or burnt area, a cancerous area or a part of the body that is affected by infection or inflammation.

Conditions which may benefit from cell therapy and may thus be treated using the method of the present invention include but are not limited to all forms of cancer including leukemia (acute or chronic), muscular dystrophy, arthritis, osteophorosis, spinal cord injuries, diabetes, myelodysplastic syndromes such as amyloidosis, myeloproliferative disorders such as Polycythemia Vera, Lymphoproliferative disorders such as Hodgkin's Disease, phagocyte disorders such as Reticular Dysgenesis, inherited metabolic disorders such as mucopolysaccharidoses, Histiocytic Disorders such as Hemophagocytosis, cardiovascular disorders, cystic fibrosis, neurological disorders such as Parkinson's, Lou Gehrig's and Alzheimer's disease, Epilepsy and Multiple Sclerosis.

Stem cells and other progenitor cells may be used, e.g. directed to areas of damage or degeneration, for repair and regeneration, or in the treatment of inflammation or cancer, and are particularly preferred for use in the methods of the invention. Treatment of any aged or injured tissues including bone, cartilage and tendon is contemplated by the present invention. Suppressive T-cells may be directed to areas affected by auto-immune disease or areas of inflammation. Cytotoxic T-cells are useful in treating cancer and CD25 and CD4 positive cells are particularly useful in dealing with inflammation. Dendritic cells can be used for treatment of infection, inflammation and arthritic and cell regeneration therapy and are of particular use in targeting lymphatic tissues in cancer patients.

Preferably the cells are from the same species as the subject and may be autologous.

The formulations and methods described herein are of use in the treatment of a wide range of conditions which can benefit from the presence of the cells themselves and/or one or more of their expressed products (typically protein products, although other secreted molecules such as nucleic acids or sugars may also be of benefit) in the target area. The treatment of these conditions by introducing therapeutically useful cells into the patient is referred to herein as “cell therapy” and thus in a further embodiment the present invention provides the use of a formulation comprising a cell and magnetic material in the manufacture of a medicament for use in cell therapy wherein the formulation is administered to a subject and part of the body of said subject is subjected to a magnetic field. Preferably, there is provided the use of a formulation comprising a cell and magnetic material in the manufacture of a medicament for the treatment of a condition selected from cancer, inflammation and infection, wherein the formulation is administered to a subject and part of the body of said subject is subjected to a magnetic field.

More preferably, there is provided the use of a formulation comprising a cell and magnetic material in the manufacture of a medicament for the treatment of an area in need of regeneration or repair such as a burn, the site of injury or a degenerate area affected by an autoimmune disease, wherein the formulation is administered to a subject and part of the body of said subject is subjected to a magnetic field.

Alternatively viewed, there is provided the use of a therapeutic formulation comprising a cell and magnetic material in the manufacture of a medicament for cell therapy, wherein the formulation is targeted to the target area through the application of a magnetic field at the target area.

The formulations will typically comprise suitable physiologically acceptable carriers and diluents for the cell type concerned e.g. saline or buffers such as phosphate buffer adjusted to around physiological pH (e.g. 7.2-7.5). It may contain sugars such as lactose or glucose and/or physiologically acceptable salts such as sodium chloride, citrate and the like. The cells may be diluted in liquid such as 5% dextrose or in saline. Suitable carriers and diluents for cell formulations are well known in the art.

As an alternative to cells, the nucleic acid containing formulation may be viral vector, e.g. an adenovirus, or a non-viral vector also capable of delivery of genetic material to a ‘host’ cell and of conjugation with magnetic material, such as a plasmid.

“Targeting” is intended to encompass both attracting a cell to the target site from a more distal location and retaining a cell at the target area. Thus in one embodiment of the present invention, the cell is administered substantially at the target site and the magnetic field serves to hold the cell in place to stop it from dispersing throughout the subject's body. In another embodiment, the cell is administered at a site that is remote from the target area and the magnetic field attracts the cell to the target area and preferably then retains the cell in the target area.

The magnetic material that is used to label the cells may be paramagnetic, supermaramagnetic or ferromagnetic. Most preferably, it will be supermaramagnetic. It will typically contain iron, nickel, cobalt, gadolinium or dysprosium or a compound, such as an oxide or alloy which contains one or more of these elements. Iron and iron compounds, such as iron oxide being especially preferred. Particularly preferred iron containing compounds are superparamagnetic iron oxide particles. Preferably, these particles are coated. The coat serves to prevent aggregation and sedimentation of the particles in aqueous solutions, achieves high biological tolerance and prevents toxic side effects. These compounds consist of nonstoichiometric microcrystalline magnetite cores, which are coated with e.g. dextrans (in ferumoxides) or siloxanes (in ferumoxsils).

Examples of particularly preferred super paramagnetic iron oxide particles are carboxydextran-coated particles (Ferrixan), sold by Schering AG under the name Resovist®, silicone-coated particles (ferumoxsil) sold e.g. under the trade name GastroMARK® or Lumirem®, dextran coated particles (ferumoxide) sold under the trade names Feridex® or Endorem®, and Ferumoxtran, sold e.g. under the trade names Combidex® or Sinerem®. Carboxydextran-coated particles are most preferred. Others include magnetodendrimers, superparamagnetic nanoparticles comprising an organic polymer and nanoparticles of a magnetic iron oxide.

The use of such particles e.g. as contrast agents in magnetic resonance imaging is well known in the art and such particles are thus known to be safe and are commercially widely available.

Methods for introducing compounds such as the magnetic materials discussed above into cells are known in the art. Typically it would be sufficient to incubate the cells with the magnetic material, e.g. for 20 mins to 4 hours at a temperature between around room temperature and around body temperature, around body temperature being preferred. For example it has been shown that CD34+ cells (10⁶ and 5×10⁵) incubated with 0.2 mmol Resovist® for 2 hours at 37° took up the magnetic material, as demonstrated by magnetic resonance imaging.

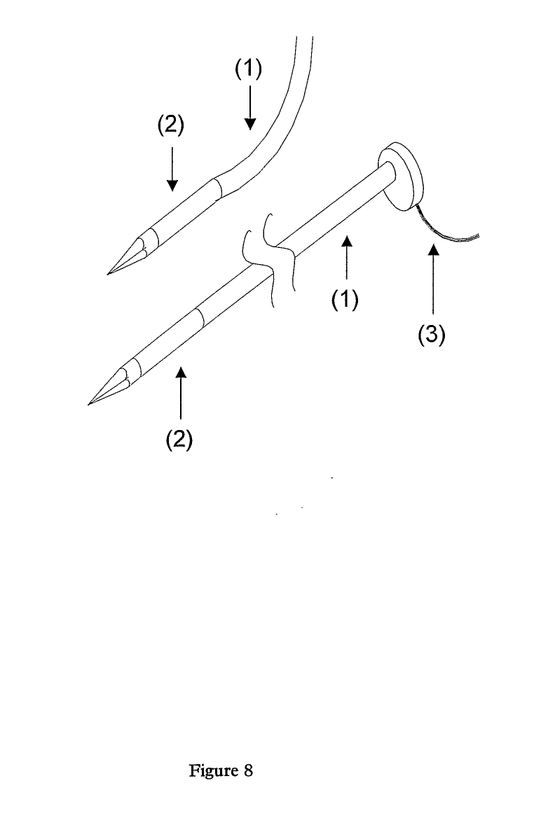

Numerous methods of generating localised magnetic fields are described herein and shown in FIGS. 8-20. The part of the body under the influence of the magnetic field will most preferably have a diameter of about 3 cm, but its size may vary depending on the application. The diameter of the magnetic field will typically not exceed 30 cm, preferably it should be less than 20 cm, more preferably less than 10 cm. The part of the body subjected to the magnetic field is never the whole body, typically it is not more than 30% of the body, usually much less, e.g. less than 20%, preferably less than 10%, most preferably less than 5%, measured on a volume or surface area basis. Thus methods in which a magnetic field is applied to the whole body of the subject are not contemplated by the present invention. The magnetic field used in the present invention is focused and localised.

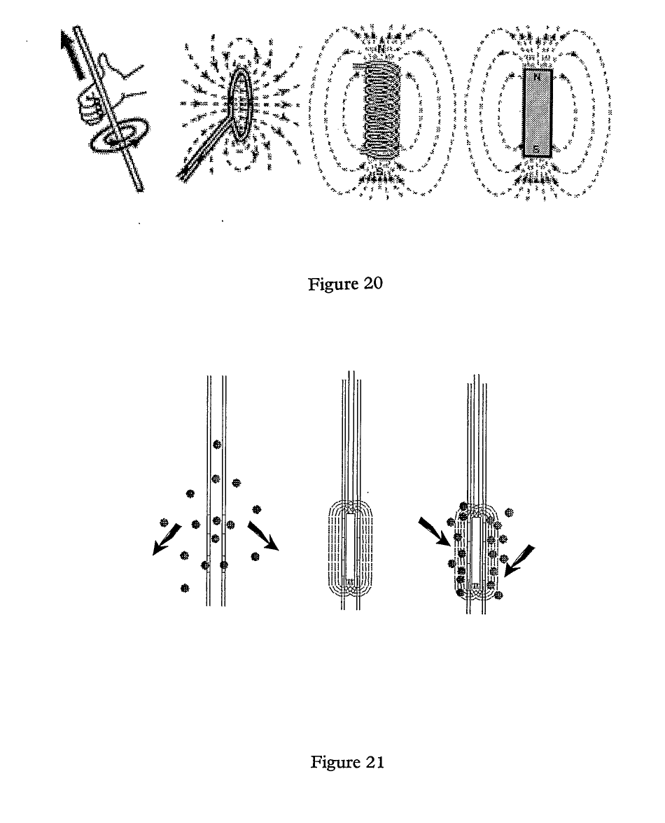

Magnets for use in the present invention may be either permanent or electromagnetic. Any of the four main magnetic field sources, current in wire, loop of wire, solenoid and bar magnet can be used in the present invention. The principles are shown in FIG. 20. The permanent magnet may be in the form of a ferrofluid contained in a sealed container. A permanent or non-permanent magnet may be introduced into the patient or placed onto his body to provide a localised magnetic field within or around the target region.

Preferred methods involve devices which include a magnet (which may be an electromagnet) within a needle (solid or hollow core) , probe, stent or catheter which can be inserted into the target region of the body. Such items may have a ferrite core with a rare earth magnet located outside the body. Electromagnetic devices may have a ferrite core with a direct current (DC) coil wound around it. Application of DC energises the coil, inducing a magnetic field. The presence or absence of the magnetic field and its strength can thus be controlled via the current. The core may have several injection ports through which the formulation and/or other fluids may be administered. A stent may be used which incorporates a magnet, for example a prostatic stent to treat patients with prostate diseases such as prostate cancer, or other stents for treating diseases affecting the pancreas or gastro-intestinal tract. Such stents may be used in treating inflammation, infection, restenosis, tissue rejection and cardiovascular disease as well as cell regeneration.

Particularly preferred in the context of the present invention are needle probes with an electromagnetic coil. Such probes have a hollow shaft which may be rigid or flexible and has a small diameter. The shaft itself is non-conductive, thus allowing the magnetic field to the focused. Such as device allows delivery of the formulation of the present invention to the target area. Subsequently, the needle probe is maintained at or in the vicinity of the target area and a current is applied to induce a magnetic field. Alternatively, a device with a hollow shaft may be used to administer the formulation to the subject and following administration, a permanent magnet is inserted into the lumen of the shaft (FIG. 21).

In a preferred embodiment, the magnetic device also contains an ultrasound array to allow for the imaging of the tissue of the subject. It also allows for monitoring of the formulation.

The magnetic devices for use in the present invention may have an outer sleeve for added stability and biocompatability. Additional features such as a balloon, pressure sensor and the like may form part of or be connected to any of the devices described above.

Thus in a further aspect, the present invention provides a medical device for targeting a formulation comprising cells and a magnetic material to part of a subject, said device comprising a magnet. Preferred features of the device are described above and below with reference to the Figures. Suitable devices include needle probes, stents, catheters etc. The device will typically have a lumen (a ribbon stent can be considered to provide a lumen). Most preferably, said device is suitable for use in the methods of the present invention.

Alternatively the magnet may be external to the body of the patient and thus be applied as, or as part of, a ring, bracelet, necklace, collar, strap, anklet or other band, e.g. to provide a magnetic field in the area of a joint, or placed on or attached to the skin of a patient as part of a ribbon, sheath, pad, bandage, sock, glove, hat etc. Low frequency electromagnets may offer deeper penetration. In this way arthritic joints or malignant melanomas may conveniently be treated, as well as muscle and general orthopaedic and spinal therapies. In one preferred embodiment a surface magnet is provided as part of a device which has a hole (i.e. lumen) through which the cell containing formulation may be administered into the subject, typically via injection. Thus a magnetic pad is provided, e.g. which comprises a substantially circular magnet, arranged such that a shaft, e.g. of a needle can be passed through the hollow center of the magnet.

In one preferred embodiment the device is coated with a magnetic layer or incorporates a magnetic lining. These will be permanent magnets. In this way a significant part of the overall device is magnetised as opposed to incorporating a small discrete magnet, although techniques can be used to coat or line certain regions of the device. One way of forming such coated devices is as follows:

Nano spheres of nickel coated NdFeB are injected via a forced jet in to a stream of atomised polymer or carrier. The static build up within the polymer encourages the fluid polymer to “wet” the nano particles. The coated particles are then sprayed directly on to the required substrate. This can be assisted by a plasma field or corona discharge. A magnetic field is applied encouraging the nano particle to migrate through the polymer and key to the substrate surface. The polymer is cured using evaporation, heat, UV, or other suitable method. The polymer carrier encapsulates the nano particles forming a flexible surface coating. The use of silicone or hydrogel as the carrier forms a “non stick” coating that avoids the labeled cells sticking to the coated surface.

In a further aspect the invention provides a medical device, e.g. a stent, catheter or needle probe, having a coating or lining extending over a part thereof which incorporates a magnetically loaded polymer. A ‘magnetically loaded polymer’ is a polymer in intimate association with magnetic particles. Alternatively said device may be made up, in whole or in part of a magnetically loaded polymer.

Various parts of the body to which it may be desired to target delivery of the therapeutic formulation have been mentioned above. The part may typically be an organ or part thereof, e.g. liver, pancreas, kidney etc. or a joint or more general region which may be affected by disease, inflammation, wounding e.g. a burn etc.

It will be appreciated that the methods of the present invention, as well as having therapeutic applications could be used in cosmetic applications. Thus, in a further aspect, the present invention provides a method in which a formulation comprising cells and a magnetic material is administered to a subject and part of the body of said subject is subjected to a magnetic field.

For cosmetic applications the target area may comprise, for example, an area of the skin where a change in e.g. pigmentation, hair growth, wrinkles or the like is desired (the introduction of stem cells in skin can produce hair). The cosmetic benefit may arise from the properties of the cells, or any products produced by the cells. For example, follicle cells may be targeted to areas where increased hair growth is desired. Collagen and/or hyaluronan-producing cells may be injected into an area which it is desired to plump up, or to smooth out wrinkles and the like.

The methods of the present invention can be practiced on any animal subject. Mammalian subjects are preferred and humans are most particularly preferred.

In a further aspect, the present invention provides a kit for targeted delivery of a cell, comprising
a) a cell labeled with a magnetic material
b) a magnetic source

Further preferred embodiments of the present invention are shown in the following Examples and Figures in which:

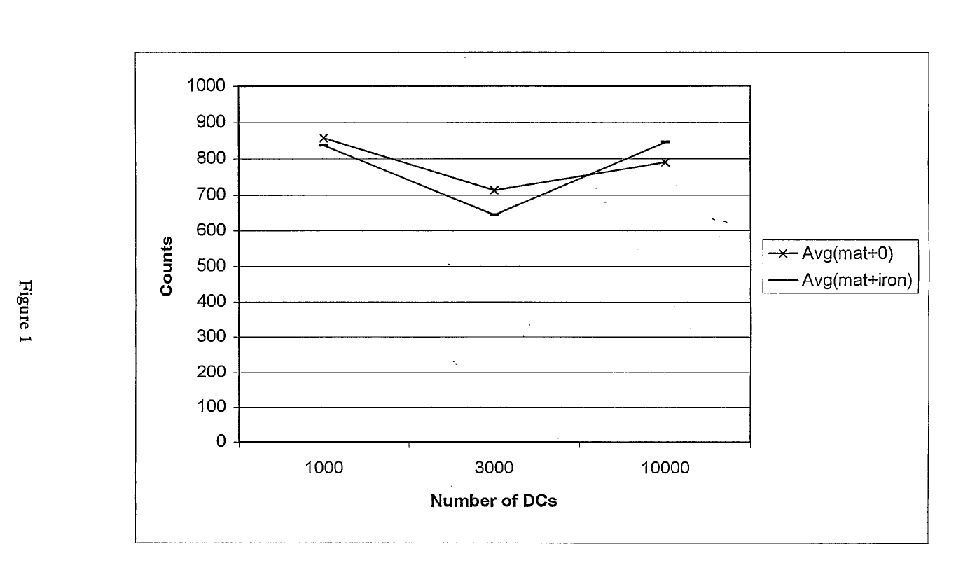

FIG. 1: The toxicity of iron dioxide nanoparticles was tested on mature dendritic cells using the Trypan blue exclusion assay.

Cells were incubated with 0.2 mmol superparamagnetic iron dioxide particles. Cell growth of labeled and unlabelled (control) cells was measured. Growth was not affected by the iron dioxide particles and no significant toxicity was observed. The number of dendritic cells cultured in the presence or absence of iron dioxide was plotted against the trypan blue counts.

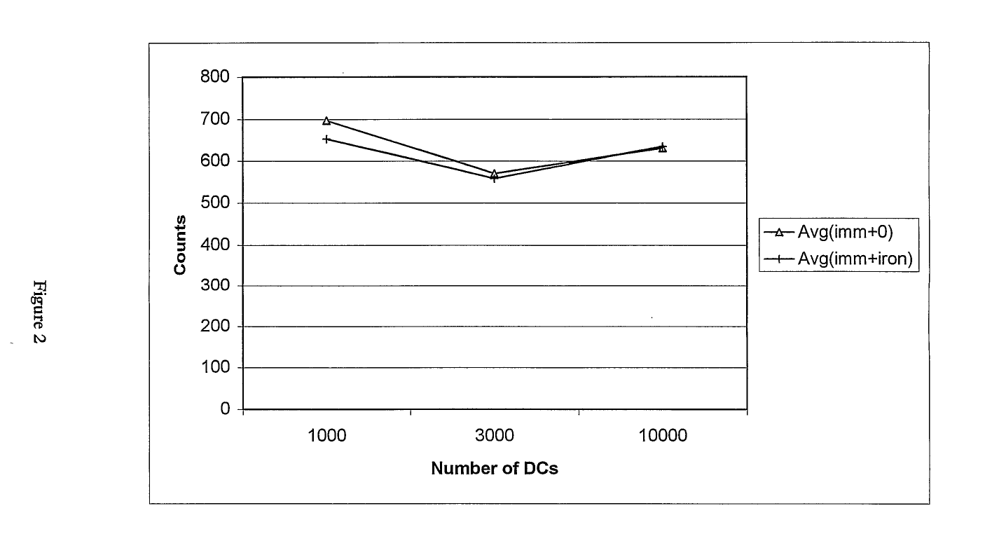

FIG. 2: The toxicity of iron dioxide nanoparticles was tested on immature dendritic cells as discussed for FIG. 1.

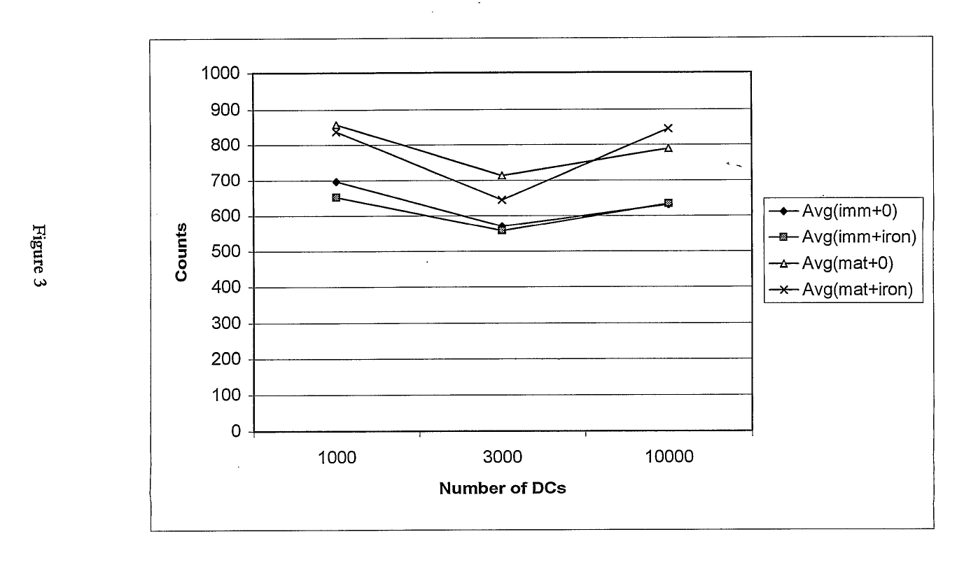

FIG. 3: This graph summarises the results of the previous two Figures.

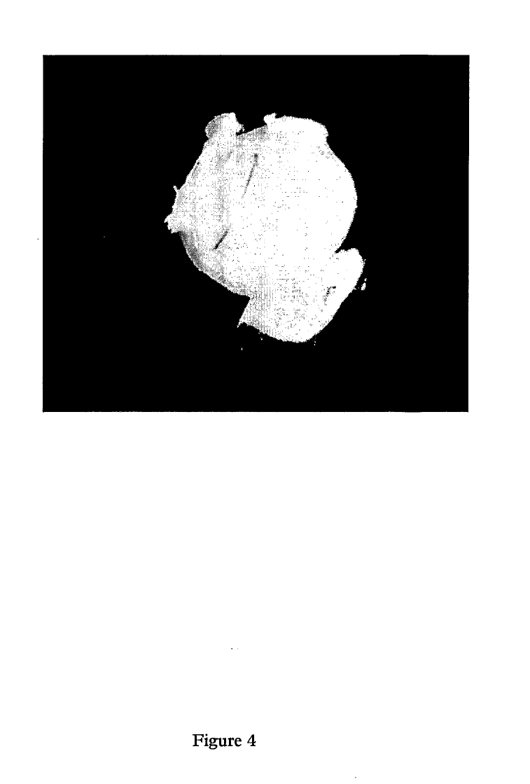

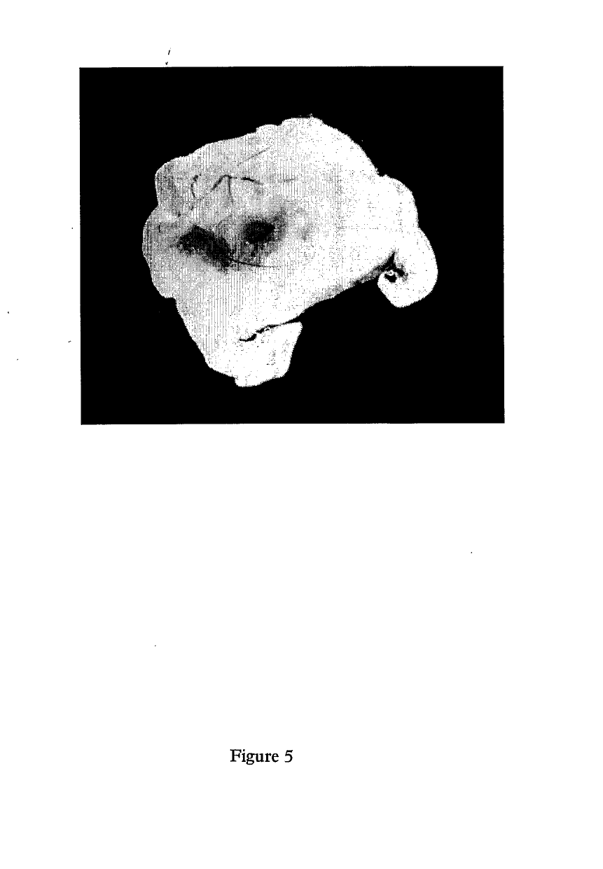

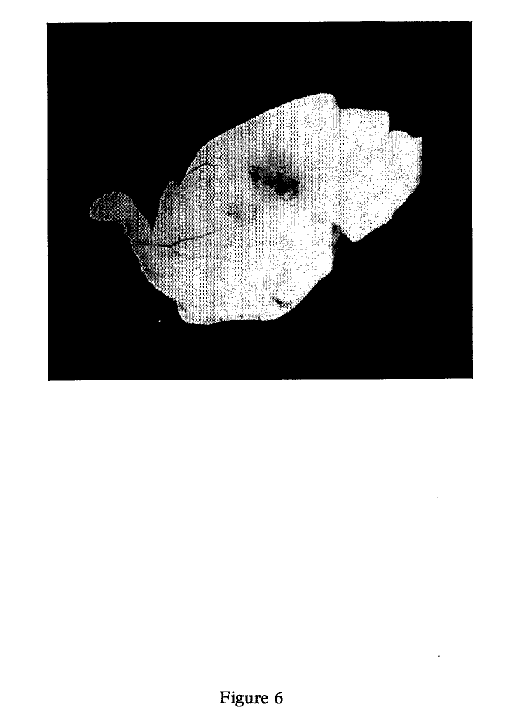

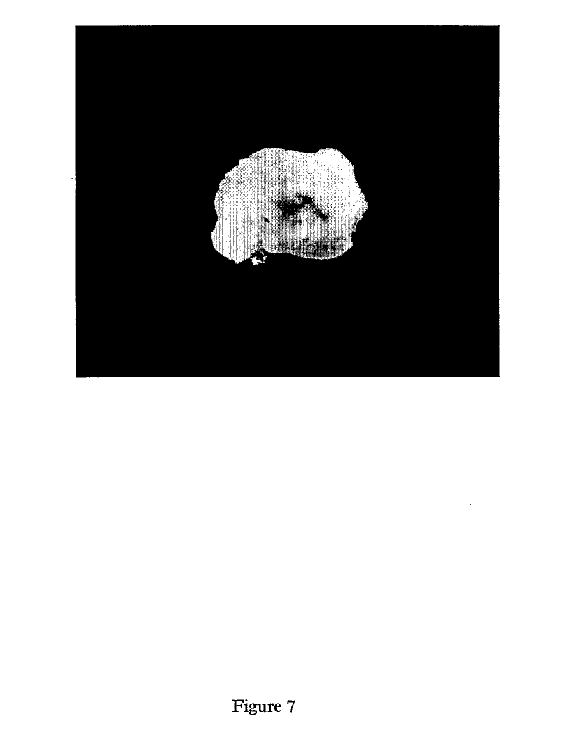

FIGS. 4-7: Photographs of a section of a mouse injected with cells labeled with iron dioxide nanoparticles (Example 2). The mouse tissue is of a light colour and the injected cells of a darker colour.

FIG. 8: This shows Examples of magnetic needle probes contemplated for use in the methods of the present invention. These probes have a shaft (1) with a small diameter which may be rigid or flexible. They may contain an electromagnet or a permanent magnet in the tip section (2). A luer and cables (3) for current supply may be present.

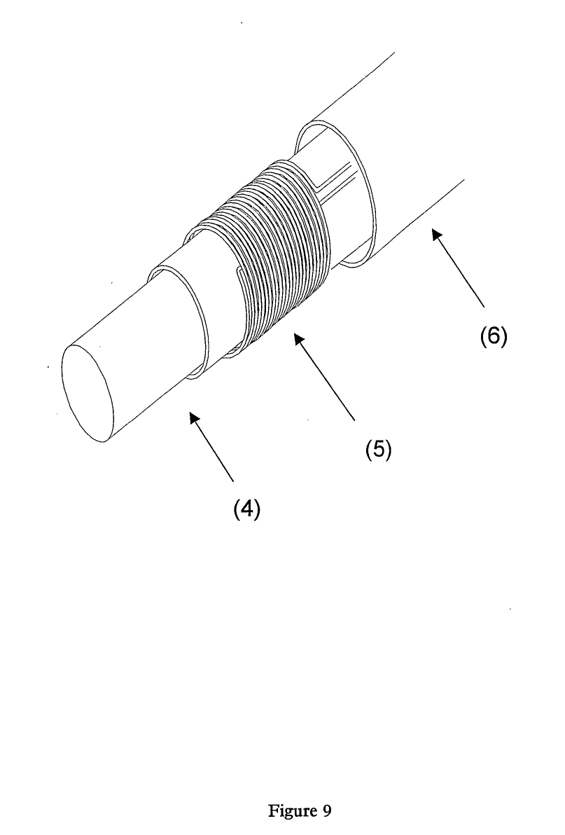

FIG. 9: Example of an electromagnetic needle probe. It has a ferrite core (4) around which a DC coil (5) is wound and an outer sleeve (6).

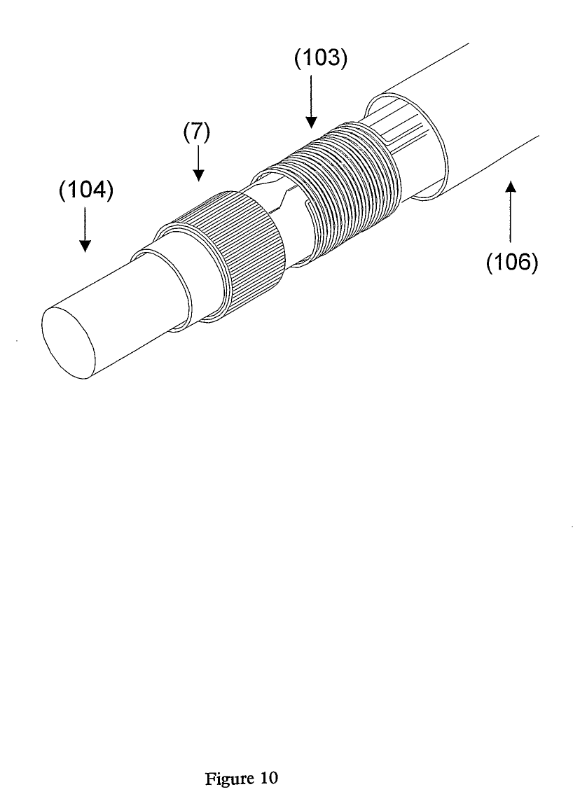

FIG. 10: Example of an electromagnetic needle probe with a ferrite core (104) around which a DC coil (105 ) is wound. It also has an ultrasound array (7) for tissue imaging and an outer sleeve (106).

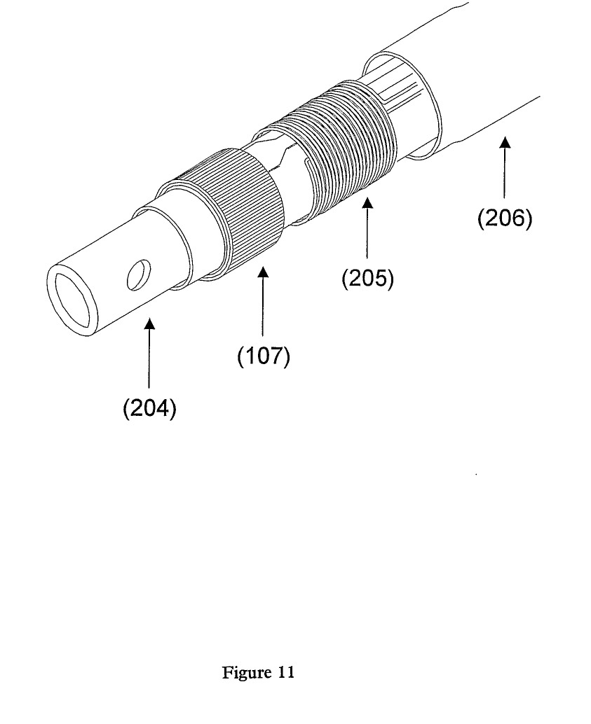

FIG. 11: Example of an electromagnetic needle probe with a ferrite core (204) which has been drilled to allow injection of fluid around which a DC coil (205) is wound. It also has an ultrasound array (107) for tissue imaging and an outer sleeve (206).

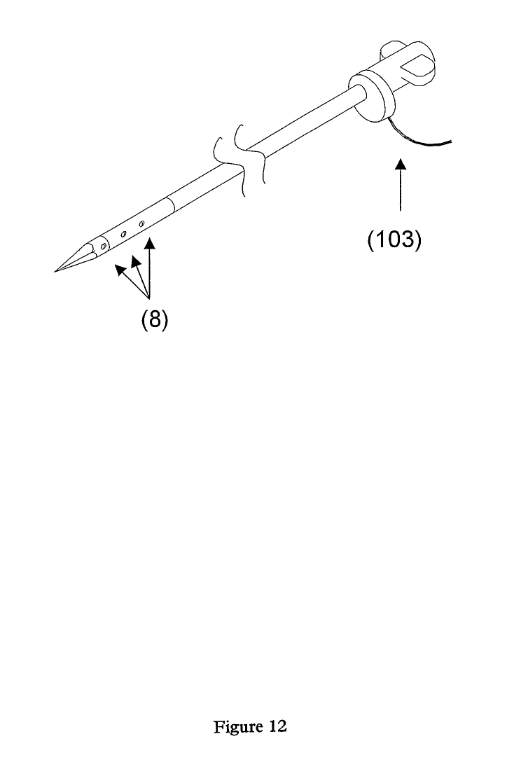

FIG. 12: Example of an electromagnetic needle probe with injection ports (8) which allow for injection of fluids such as contrast materials. It has a luer and cable (103) for current supply.

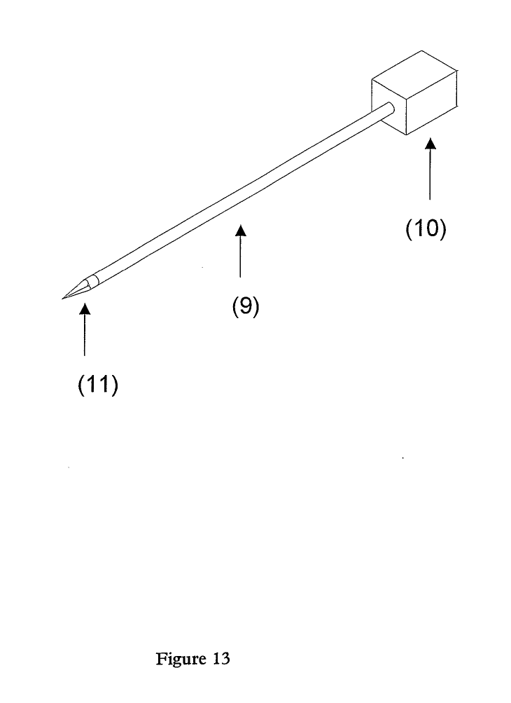

FIG. 13: Example of a permanent magnetic needle probe which has a ferrite core in an outer sleeve (9) and a rare earth magnet (10) which transfers the magnetic field along the ferrite core to the tip section (11).

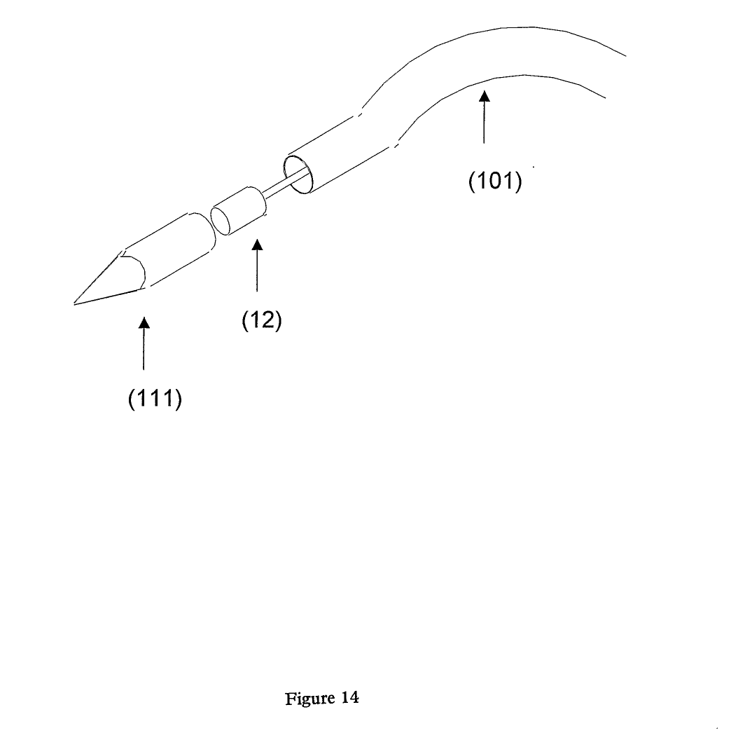

FIG. 14: Example of a permanent magnetic needle probe which has a permanent magnet in the form of a rare earth magnet or ferrofluids cell (12) a shaft (101) and a tip (111). Ferrofluids may be used by sealing them iri a capsule or tubing section.

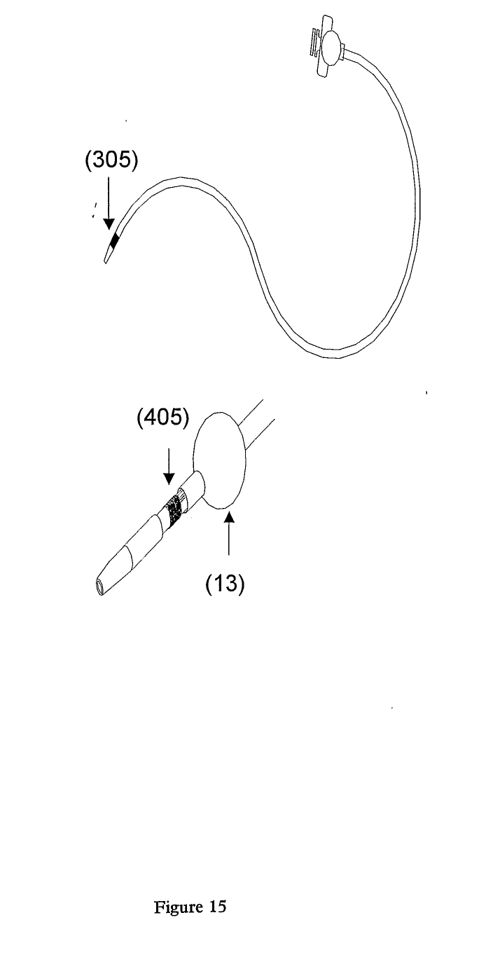

FIG. 15: Examples of magnetic catheters with a DC coil (305) and (405). These can be adapted for all uses generally contemplated for a catheter such as urinary and venous catheters. Additional features such as a balloon (13) and/or pressure sensors may be present.

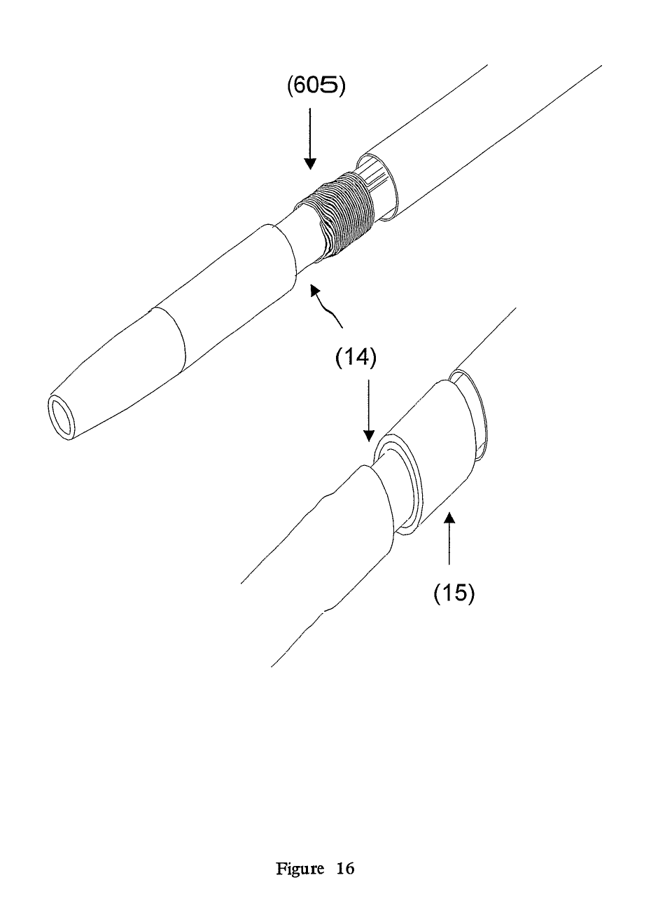

FIG. 16: Further examples of urinary or venous catheters with a DC coil (605) or a rare magnet mounted on the catheter shaft (14). The magnet may be covered with a sleeve or over moulded (15).

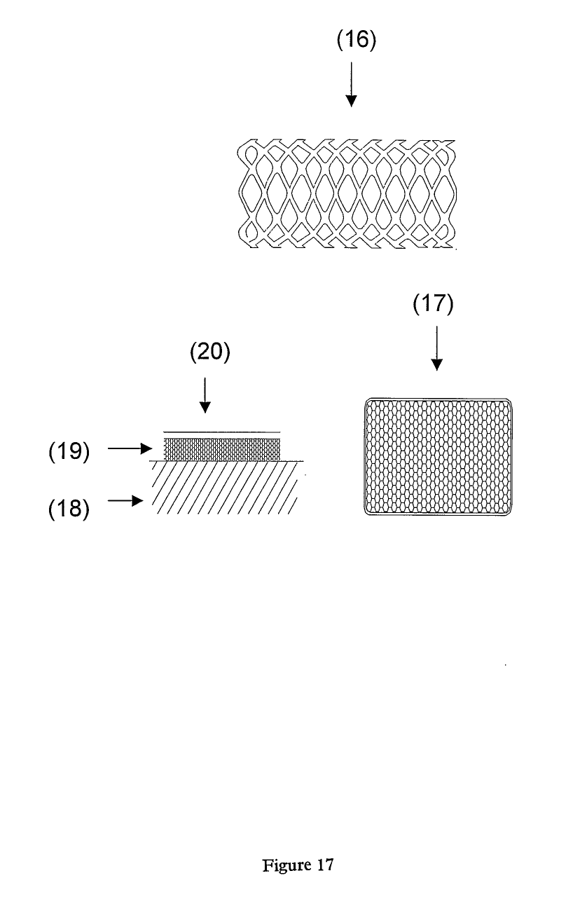

FIG. 17: This Figure illustrates how devices such as stents may be coated with a magnetic layer. Magnetic nano spheres allow the coating of stents (16) made e.g. of nitinol or stainless steel for e.g. cardiology and peripheral systems. Magnetically loaded polymers can be fabricated into stent or linings, forming a completely magnetic structure (17). An Example of a stent strut (18) with a layer of nano spheres (19) and a coating layer (20) is shown.

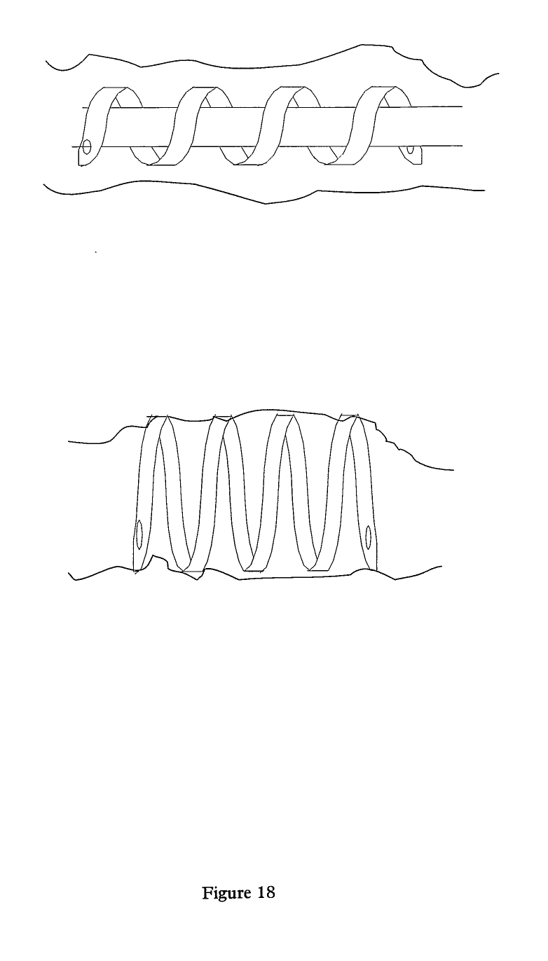

FIG. 18: Magnetic polymers may be used to prepare ribbon stents. The upper part of the figure shows the ribbon stent and the delivery shaft holding the stent flat; after the shaft is removed the ribbon stent expands to hold open the lumen of the blood vessel or equivalent, as shown below.

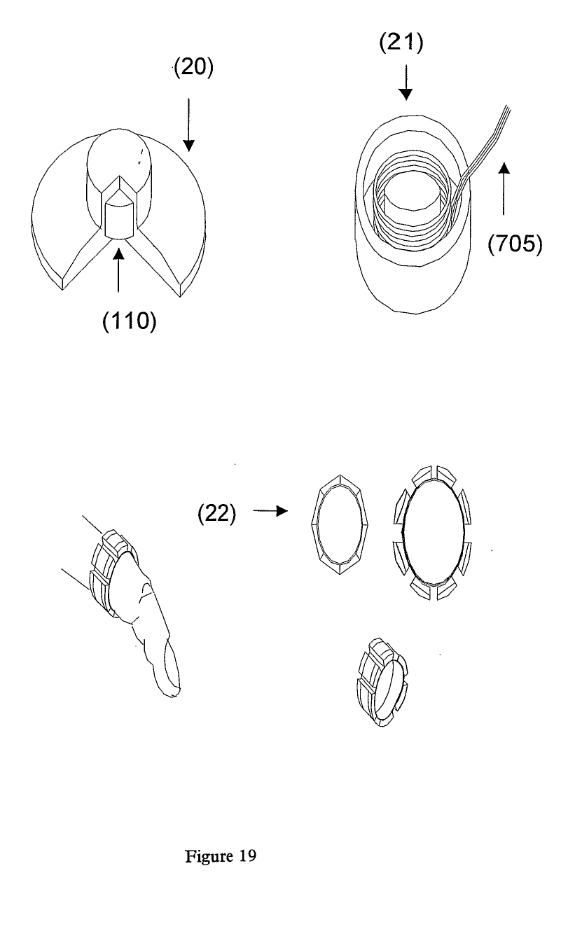

FIG. 19: Examples of external magnets which can be placed onto the skin of the subject. A rare earth magnet (110) or coil (705) may be moulded into a reusable pad (20) or (21). The magnets may be formed into belts, rings, collars and the like. This Figure shows Examples of magnetic rings (22).

FIG. 20: This figure illustrates the four main magnetic field sources. From left to right these are: current in wire, loop of wire, solenoid and bar magnet.

FIG. 21: This Figure illustrates an Example of carrying out the method of the present invention. A device with a hollow core is used to administer the formulation to the subject (left). A permanent magnet is inserted into the lumen of the device (middle). The magnetic field generated by the magnet attracts the formulation and retains it in the vicinity of the device (right).

### EXAMPLES

### Example 1

### In vitro Assay

CD34+ cells (10 ⁶) were labeled with superparamagnetic iron oxide (SPIO) particles. Cells were incubated with 0.25 mmol commercially available Resovist (the active ingredients are SPIOs) for 2 hours at 37° C. Labeled cells were placed on plastic Petri dish, which contained either charged or mock magnet and their behaviour was observed by microscope. The charged magnet heavily attracted labeled cells and its surface was completely covered by them in only 5 minutes. No such phenomenon was observed using the mock magnet.

The in vitro toxicity of Resovist was tested by Trypan blue exclusion assay and proved to be non significant (<4%) . This is also shown in FIGS. 1-3.

### Example 2

### In vivo Assay

Cells labeled as described in Example 1 were injected subcutaneously under the flank into one control and one experimental mouse. A N32 Neodymium Iron Boron solid disc magnet was applied to the skin of the experimental mouse near the injection site; no magnet was applied to the control mouse. After 1 hour the area of skin around the injection site was excised from both mice. In the experimental mouse (i.e. the mouse to which a magnetic field had been applied) the labeled cells were retained at the injection site (shown in FIGS. 4-7) but cells were dispersed from the injection site in the control mouse. This result demonstrates that that labeled cells can be retained at desired locations by the action of a magnet.

1. A method in which a formulation comprising cells and a magnetic material is administered to a subject and part of the body of said subject is subjected to a magnetic field.

2. Use of a formulation comprising a cell and magnetic material in the manufacture of a medicament for use in cell therapy wherein the formulation is administered to a subject and part of the body of said subject is subjected to a magnetic field.

3. Use according to claim 2 wherein the condition which is treated by the cell therapy is selected from cancer, inflammation and infection.

4. Use according to claim 2 wherein the cell therapy is directed to an area in need of regeneration or repair such as a burn, the site of injury or a degenerate area affected by an autoimmune disease.

5. A method as claimed in claim 1 wherein the cells contain magnetic material.

6. A method according to claim 1 wherein the magnet that generates said magnetic field is in direct or indirect physical contact with the subject.

7. A method according to claim 1 wherein the cell is a stem cell.

8. A method according to claim 1 wherein the part of the body subjected to the magnetic field is less than 30%, preferably less than 10% of the whole body.

9. A method according to claim 1 wherein the magnetic field has a diameter of less than 20 cm.

10. A method according to claim 1 wherein the magnetic material is a superparamagnetic iron oxide particle.

11. A method according to claim 1 wherein the subject is a human subject.

12. A cosmetic method in which a formulation comprising cells and a magnetic material is administered to a subject and part of the body of said subject is subjected to a magnetic field.

13. A medical device for targeting a formulation comprising cells and a magnetic material to part of a subject, said device comprising a magnet.

14. (canceled)

15. A stent, catheter or needle probe comprising or being coated or lined with a magnetically loaded polymer.

16. A method according to claim 1, wherein a medical device for targeting the formulation comprising cells and a magnetic material is used to target the formulation to a part of a subject.
